(19) United States
(12) Patent Application Publication
(10) Pub. No.: US 2004/0171694 A1
(43) Pub. Date: Sep. 2, 2004

(54) Dietetic preparation and use of an alpha-hydroxy carboxylic acid(citric acid for the treatment of obesity

(76) Inventors: Katrien Maria Jozefa Van Laere, Heteren (NL); Rene John Raggers, Amsterdam (NL)

Correspondence Address: Young & Thompson, Second Floor, 745 South 23rd Street, Arlington, VA, 22202, US

(21) Appl. No.: 10/480,487

(22) PCT Filed: Jun. 14, 2002
(86) PCT No.: PCT/NL02/00394

(51) Int. Cl. A61K 31/19

(52) U.S. Cl. 514/574

(57) ABSTRACT

Method of inhibiting intestinal carbohydrate absorption in mammals and a dietetic preparation for use in such a method. The method comprises orally administering a dietetic preparation to such mammal, the preparation containing a-hydroxy carboxylic acid component, whose intestinal absorption is sodium dependent, in an amount equivalent to at least 1 wt. % citric acid, so as to provide a-hydroxy carboxylic acid component in an amount which is therapeutically effective to achieve inhibition of intestinal absorption of carbohydrate. The dietetic preparation is in the form of an oral dosage unit of between 0.1 and 100 grams, and contains between 2 and 90 wt. % of a-hydroxy carboxylic acid component, between 1 and 80 wt. % of a carbohydrate absorption inhibitor and between 97 and 9 wt. % of pharmaceutically acceptable excipient.

### TECHNICAL FIELD

This invention relates to a method of inhibiting intestinal carbohydrate absorption in mammals and a dietetic preparation for use in such a method. More particularly the present invention is concerned with the administration of α-hydroxy carboxylic acid component, whose intestinal absorption is sodium dependent, in an amount effective to achieve inhibition of intestinal carbohydrate absorption. The α-hydroxy carboxylic acids used in accordance with the invention can be represented by the general formula R—C(OH)COOH—R. Citric acid is an example of an α-hydroxy carboxylic acid which may suitably be employed in the present method.

The present invention also concerns a dietetic preparation in the form of an oral dosage unit of between 0.1 and 100 grams, said preparation containing between 2 and 90 wt. % of α-hydroxy carboxylic acid component, whose intestinal absorption is sodium dependent, between 1 and 80 wt. % of a carbohydrate absorption inhibitor selected from the group consisting of polyphenols, gymnemic acid and mixtures thereof, and pharmaceutically acceptable excipient.

### BACKGROUND OF THE INVENTION

Reduction of carbohydrate absorption in the intestine of animals, especially humans, is nutritionally and medically of great importance. Reduction of absorption can for example facilitate body weight management, e.g. as part of a method of treating obesity, and can be advantageous for subjects suffering form diabetes or hypoglycaemic state.

Reduced carbohydrate absorption by the intestine is thought to reduce fat formation. In a normal diet many carbohydrate containing components are present. During digestion of the carbohydrates, monosaccharides, e.g. glucose, will be formed which can be readily absorbed by the intestine. The absorbed glucose can subsequently be converted to water and carbon dioxide, glycogen, glycol or fatty acids, the last predominantly occurring when an excess of glucose is present, e.g. when a vast amount of carbohydrates is consumed.

Many preparations have been proposed to reduce carbohydrate digestion either alone or in combination with components capable of reducing absorption of the carbohydrates in the intestine. Such compositions will ultimately result in the reduced in vivo availability of glucose, thereby reducing the formation of adipose tissue, contributing to weight loss, reducing blood glucose levels, decreasing fluctuations in blood glucose levels etc. These effects are advantageous for e.g. obese or diabetic subjects and subjects having the desire to maintain a low weight or desirable silhouette.

Reduction of carbohydrate digestion can for example be accomplished by the ingestion of components capable of reducing digestive enzyme activity, e.g. by reducing pancreatic amylase and α-glucosidase activity.

α-Glucosidase converts non-absorbable dietary starch and sucrose into absorbable monosaccharides. Inhibitors of α-glucosidase inhibit such conversion, resulting in the delay of formation and absorption of monosaccharides. Therefore, these inhibitors reduce the concentration of post-prandial blood glucose.

An amylase inhibitor reduces the activity of human pancreatic α-amylase and moderates the digestion of ingested starch by inhibiting the conversion of carbohydrates into smaller carbohydrate polymers, thus inhibiting an increase in blood glucose level and reducing insulin secretion.

U.S. Pat. No. 5,840,705 discloses an α-glucosidase inhibitor mildly inhibiting alpha-glucosidase locally present in the micro-villus of the small intestine. The inhibitor delays the digestion of starch, starch-derived oligosaccharides and sucrose, so that the inhibitor has an action of suppressing rapid increase in blood glucose level and an action of suppressing insulin secretion at a lower level.

U.S. Pat. No. 6,174,904 discloses a method for treating glycometabolism disorders in a mammal in need thereof, which comprises administering to such mammal a therapeutically effective amount of an insulin sensitivity enhancer in combination with an α-glucosidase inhibitor, wherein said α-glucosidase inhibitor can be acarbose, voglibose and miglitol, and the insuline sensitivity enhancer can be troglitazone.

JP2000103742 discloses an α-amylase inhibitor obtained from extracts of Gambir, a material obtained from the root of Sassafras albidum, having high safety and capable of suppressing absorption of carbohydrates and preventing obesity, diabetes, or the like.

Reduction of absorption of glucose in the intestine can for example be accomplished by intestinal absorption reducing components such as gymnemic acid, which can be extracted from Gymnema sylvestre as reported by Shimizu et al. “Suppression of glucose absorption by some fractions extracted from Gymnema sylvestre leaves”, J. Vet Med. Sci (1997), 59(4), 245-251.

Combinations of components capable of reducing the activity of intestinal carbohydrate degrading enzymes and components which reduce the intestinal absorption of glucose are for example described in WO0117369, which discloses the combination of α-amylase inhibitors, for example plant protein derived α-amylase inhibitors and absorption inhibitors e.g. inulin and fructo-oligosaccharides.

Although many of the compositions reducing carbohydrate absorption are know in the art and available on the market, still such compositions are open to improvements, particularly because of undesirable side effects occurring when administering these compositions in substantial amounts. Commercially available compositions that include effective enzyme inhibitors and/or components which reduce intestinal glucose absorption are found to cause insufficient water uptake, potentially resulting in dehydration. Glucose is co-transported over the intestinal wall with salt, and thus fulfils the important role of increasing the cellular concentration of salt within the intestine and inducing osmotic water transport from the intestine to the cells. Reduced glucose transport, e.g. due to reduced availability of glucose or inhibition of carbohydrase enzymes, will result in reduced water transport. The resulting reduction in water absorption is a common and undesirable side effect of existing compositions comprising carbohydrase inhibiting components and/or glucose absorption inhibiting components. The decreased water uptake observed for these compositions often leads to increased excretion of water in the faeces, a cause of diarrhea and other adverse effects. Thus the need for a potent and safe amylase inhibitor and/or glucosidase inhibitor which can suitably be used in compositions which reduce glucose absorption in the intestine is well recognised in the art.

Another drawback of many compositions currently available suitable for the purposes indicated above, is the inclusion therein of components of which no extensive safety data exist, making the use, especially long term use of such products dubious and potentially unsafe.

### SUMMARY OF THE INVENTION

Surprisingly, it was found that the administration of an effective amount of α-hydroxy carboxylic acid component, whose intestinal absorption is sodium dependent, provides a solution to the above problems. The use of such α-hydroxy carboxylic acid components fulfils a long standing need for a safe and effective method of inhibiting carbohydrate absorption, without the risks of diarrhoea and dehydration that are associated with the use of existing carbohydrase inhibitors. An example of an α-hydroxy carboxylic acid that may advantageously be used in accordance with the present invention is citric acid. Citric acid is used in many applications, particularly in the food industry. However, the use of citric acid in a method of inhibiting intestinal carbohydrate absorption is not disclosed in the prior art.

U.S. Pat. No. 4,689,219 describes oral pharmaceutical compositions in dry powder or granular form adapted to be added to water or a drink for treatment of diabetes, which compositions comprise xanthan gum and locust bean gum as well as 2.5 to 10 wt. % of an organic acid such as citric acid. The combination of xanthan gum and locust bean gum is said to have an inhibitory effect on the diffusion of glucose across a membrane. The organic acid is included to control the rate of gelation of the mixture of the 2 aforementioned gums.

A nutritional tea beverage currently on the market under the name “Herbal Slimmer” from Tribal Tonics™ comprises green tea extract, other herbal extracts and citric acid. The product has a high content of carbohydrates. Another nutritional beverage product “Over 30™” also contains green tea and citric acid and a vast amount of digestible carbohydrates.

Hansawasdi et al, “α-Amylase Inhibitors from Roselle Tea”, Biosc. Biotechnol. Biochem. (2000), 64(5), 1041-1043 report the results of a study wherein the α-amylase inhibiting properties of tea extract were compared to that of structurally related citric acid, which is said to be a known inhibitor of fungal α-amylase.

U.S. Pat. No. 4,477,434 describes medicinal compositions, foods and beverages, comprising a combination of papain and citric acid, having therapeutic effects on diseases of the circulatory system and the digestive system. Diseases of the circulatory system are said to include diabetes, hemorrhoids, hypertension, gout. Diseases of the digestive system mentioned in the patent include hypertrophy of the liver, hepatitis and pancreatitis. The effect of citric acid on the intestinal absorption of carbohydrates it not discussed in this patent.

### DETAILED DESCRIPTION OF THE INVENTION

One aspect of the present invention relates to a method of inhibiting intestinal absorption of carbohydrates in mammals, which method comprises orally administering a dietetic preparation to such mammal, said preparation containing α-hydroxy carboxylic acid component, whose intestinal absorption is sodium dependent, in an amount equivalent to at least 1 wt. % citric acid, so as to provide α-hydroxy carboxylic acid component in an amount which is therapeutically effective to achieve inhibition of intestinal absorption of carbohydrate.

The term “inhibition” should not be interpreted restrictively, i.e. in the context of this application it encompasses prevention as well as suppression (or reduction) of intestinal absorption of carbohydrate, in particular as a result of carbohydrase inhibition. An α-hydroxy carboxylic acid is a carboxylic acid wherein the α-carbon atom is substituted with a hydroxy group. These acids can be represented by the general formula:

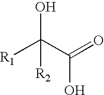

wherein R₁ and R₂ are independently selected from a hydrogen atom, a C₁-C₅ alkyl group, a C₆-C₁₂ aryl group, a heterocyclic C₆-C₁₂ cycloalkyl- or -arylgroup, a carboxylic group or a —CH₂COOH group. Preferably R₁ and R₂ are independently selected from a hydrogen atom, a C₁-C₅ alkyl group, a carboxylic group or a —CH₂COOH group. The α-hydroxy carboxylic acids employed are most effective if they contain less than 12 carbon atoms, preferably between 3 and 10 carbon atoms, more preferably between 3 and 6 carbon atoms. In addition the total number of hydroxyl groups preferably does not exceed 4.

The term α-hydroxy carboxylic acid component encompasses the α-hydroxy carboxylic acid itself, precursors of said acid and metabolites of the acid which display a similar inhibiting functionality. The term mammals includes all warm blooded vertebrates. Preferably the present method is applied to humans or pets such as dog, cat and rabbit.

Whether or not the absorption of a specific α-hydroxy carboxylic acid is sodium dependent can be determined in an in vitro model of epithelium cells lining the intestinal tract. Such methods are well known in the art and often performed in so called Ussing chambers. Sodium dependency of absorption may also be determined by an in vivo marker perfusion technique as described by Patra et al., “Enhanced sodium absorption by citrate: an in vivo perfusion study of rat small intestine”, J. Pedriatr Gastoenterol Nurt (1990) 11, 385-388.

The discovery by the current inventors that α-hydroxy carboxylic acids whose intestinal absorption is sodium dependent, not only stimulates rehydration, thereby preventing dehydration, but also inhibits intestinal α-amylase, has made it possible to develop a method of inhibiting intestinal carbohydrate absorption which method does not suffer from the adverse side effects normally occurring in known compositions having such action.

Glucose is absorbed in the gastrointestinal tract in a sodium dependent manner causig hydration of the intestinal cells. In the absence of high concentrations of glucose and other monosaccharides, e.g. as a result of inhibition of intestinal carbohydrase enzymes, the sodium uptake is reduced, resulting in a decreased water absorption from the gastrointestinal tract, potentially followed by adverse side effects such as diarrhoea. The α-hydroxy carboxylic acid component used in the present method is absorbed in the gastrointestinal tract in a sodium dependent way, thereby increasing the sodium concentration of the gastrointestinal cells. The increased cellular sodium concentration will increase the intracellular osmotic value, which again will induce intestinal water absorption, i.e. rehydration. Thus the α-hydroxy carboxylic acid component which is absorbed in a sodium dependent way offers the advantage that it counteracts the reductions of sodium absorption induced by the carbohydrase inhibiting action of the present dietetic preparation.

Inhibition of digestive enzymes often results in the excretion of intestinal fluid in the faeces, e.g. in the form of diarrhoea, resulting in a loss of intestinal acidic compounds and intestinal water. The loss of intestinal acidic compounds increases the pH of the intestine, resulting in several adverse side effects, such as cellular damage to the digestive tract and inhibition of conversion of proenzyme pepsinogen to pepsin, which subsequently interferes with protein breakdown. Additionally, a rise of the intestinal pH stimulates the proliferation and growth of pathogenic bacteria in the digestive tract, such as Escherichia coli, Clostridium species and Bacterioides. Generally, the pathogenic bacteria are known to grow in the intestine when the pH is in the range of 5 or more, whereas the bacteria are inhibited at a pH in the range of 3.6 or below. Oral administration of α-hydroxy carboxylic acid component whose absorption is sodium dependent will minimise dehydration and will thus prevent or suppress the proliferation of intestinal pathogenic bacteria caused by the inhibition of intestinal carbohydrase enzymes.

Without wishing to be bound by theory, the inventors believe that the present α-hydroxy carboxylic acid component stimulates rehydration through the intestinal co-transport of the acid and sodium. It has been shown that, for instance, citrate uptake by brush border membranes occurs by a Na(+)-dependent transport mechanism (Wolffram et al., “Transport of tri- and dicarboxylic acids across the intestinal brush border membrane of calves”, J. Nutr, (1990), 120(7), 767-774). Furthermore, in humans, citrate has been shown to stimulate absorption of sodium and consequently water absorption from the human jejunum (Rolston et al, “Acetate and citrate stimulate water and sodium absorption in the human jejunum”, Digestion, (1986), 34(2), 101-104). The sodium co-transported with the citrate is believed to induce an increase of intracellular osmotic value, resulting in water transport from the intestine to the cells, i.e. rehydration.

The present method produces particularly good results if the α-hydroxy carboxylic acid component is provided in a daily amount equivalent to at least 0.25 mg, preferably at least 0.5 mg citric acid per kg of bodyweight of the mammal. Most preferably the α-hydroxy carbbxylic acid component is provided in a daily amount equivalent to at least 1 mg, more preferably equivalent to at least 3 mg citric acid per kg of bodyweight.

The amount of α-hydroxy carboxylic acid component which is equivalent to a given amount of citric acid can be established as follows:

1. calculate the equivalent molar amount of citric acid,

2. multiply the molar amount by a factor 3

3. divide the result of the multiplication by the number of carboxylic groups present in the α-hydroxy carboxylic acid

4. calculate for the α-hydroxy carboxylic acid component how many mg's are equivalent to the molar amount obtained from 3.

In accordance with the method of the invention, preferably the dosage form is chosen such that preparation can be administered in dosage units of between 0.025 and 200 g, more preferably between 0.1 and 100 g, and most preferably between 0.25 and 50 g.

For a human being, a single dosage unit preferably comprises α-hydroxy carboxylic acid component in an amount equivalent to at least 40 mg, more preferably at least 100 mg, most preferably above 250 mg citric acid. Meals, such as breakfast, lunch, and dinner usually contain digestible carbohydrates in amounts of 20 grams or more. According to a preferred embodiment, the dietetic preparation used in the method according to the invention is directed towards the inhibition of the absorption the digestible carbohydrates from meals. In order to obtain sufficient inhibition of carbohydrate absorption following the consumption of such a meal, the preparation preferably contains α-hydroxy carboxylic acid component in an amount equivalent to at least 150 mg, more preferably at least 200 mg citric acid. To ensure a sufficient rehydration when additional carbohydrase inhibitors or monosaccharide uptake inhibitors are present in the dietetic preparation used in the method according to the invention, the dietetic preparation preferably contains at least α-hydroxy carboxylic acid component in an amount equivalent to at least 100 mg, more preferably at least 150 mg, most preferably at least 200 mg citric acid.

If the present dietetic preparation is used to reduce the absorption of carbohydrates originating from a separately consumed foodstuff (hereinafter referred to as dietary carbohydrates), it is undesirable for said preparation to contain large amounts of digestible carbohydrates as this will counteract the objective of achieving inhibition of carbohydrate absorption. Hence, in a preferred embodiment, the dietetic preparation comprises less than 60 wt. %, more preferably less than 40 wt. %, even more preferably less than 25 wt. % and especially preferred, less than 10 wt. % digestible carbohydrates calculated on dry weight of the preparation. Unless indicated otherwise, the percentages mentioned in this application apply to the consumable part of the preparation, e.g. not including packaging material.

According to another preferred embodiment the caloric value of digestible carbohydrates is less than 50%, preferably less than 25% and more preferably less than 10% of the total caloric value of the preparation according to the invention. In yet another preferred embodiment the amount of α-hydroxy carboxylic component, calculated as citric acid equivalent, exceeds the amount of digestible carbohydrates in the preparation. More preferably the amount of α-hydroxy carboxylic component, calculated as citric acid equivalent, is at least twice, preferably at least thrice as high as the amount of digestible carbohydrates in the preparation.

Transport of glucose in a cell is accompanied by transport of Na⁺ and water absorption. Inhibition of glucose absorption will normally lead to reduced water absorption which again may give rise to diarrhoea. Although an α-hydroxy carboxylic component such as citric acid will stimulate water transport, i.e. rehydration, it is undesirable for the present preparation to contain large amounts of water as this will increase the risk of diarrhoea and other adverse side effects. Hence, in a preferred embodiment, the dietetic preparation used in the method of the invention contains less than 95 wt. %, preferably less than 90 wt. %, even more preferably less than 75 wt. % and most preferably less than 25 wt. % water.

In order for the present preparation to be effective in inhibiting carbohydrate absorption said preparation should deliver citric acid into the intestine in a rather concentrated form, i.e. at least 1% by weight of the preparation. Preferably the dietetic preparation used in the present method contains α-hydroxy carboxylic acid component in an amount equivalent to at least 2 wt. %, more preferably at least 5 wt. % and most preferably at least 8 wt. % citric acid. Generally the preparation will contain the α-hydroxy carboxylic acid component in an amount which is equivalent to less than 95 wt. % citric acid, preferably less than 90 wt. % citric acid and more preferably less than 75 wt. % citric acid.

According to a very preferred embodiment of the invention the α-hydroxy carboxylic acid component is citric acid component. The term “citric acid component” as used herein, encompasses citric acid, precursors of citric acid and metabolites of citric acid which display a similar inhibiting functionality. In case the α-hydroxy carboxylic acid component is citric acid component the amount of citric acid component which is equivalent to a given amount of citric acid is easily established by calculating which amount of the citric acid component would liberate said given amount of citric acid, assuming that the citric acid component is fully converted, i.e. releases all citric acid contained therein.

Citric acid (2-Hydroxy-1,2,3-propanetricarboxylic acid) is a naturally occurring fruit acid, produced commercially by microbial fermentation of a carbohydrate substrate is widely available, e.g. as monohydrate or anhydrous citric acid and is the most widely used organic acidulant and pH-control agent in foods, beverages, pharmaceuticals and technical applications. However, it has not been recognised before to have the advantageous capability of inhibiting carbohydrate absorption.

In Europe, citric acid monohydrate and anhydrous are listed as generally permitted food additives (E 330) and may be added to all foodstuffs. The US Food and Drug Administration (FDA) affirmed citric acid as GRAS (generally recognized as safe) and permitted the use in food according to current GMP (CFR § 182.1033), without setting an upper limit.

The dietetic preparation in accordance with the invention may suitably take the form of tablets, capsules, powders, foodstuffs (e.g. nutritional bars or desserts). According to a preferred embodiment of this invention, the α-hydroxy carboxylic acid component is ingested in the form of a tablet or capsule, having a weight between about 25 mg and 3000 mg, preferably between about 100 mg and 2500 mg, most preferably between 200 and 2000 mg. In order to prevent adverse taste effects, particularly in case the acid component is administered in a concentrated way in the form of a capsule or a tablet, said tablet or capsule is preferably coated in such a way that the acid component is not released in the mouth. Hence, in a preferred embodiment, the α-hydroxy carboxylic acid is orally administered in a solid unit dosage form wherein at least 95% of the α-hydroxy carboxylic acid reaches the stomach in solid state, more preferably at least 98%. Effectiveness of the α-hydroxy carboxylic acid component is further enhanced when the α-hydroxy carboxylic acid is specifically delivered in the small intestine, e.g. by incorporating the α-hydroxy carboxylic acid component in a tablet or capsule having a stomach acid resistant coating, e.g. coated with an acid resistant polymer, or alternatively by employing an α-hydroxy carboxylic acid precursor which releases most of the α-hydroxy carboxylic acid in the small intestine.

In a preferred embodiment of the present method the α-hydroxy carboxylic acid is delivered in the intestine in a largely protonated form. Thus, preferably at least 50%, more preferably at least 75% of the α-hydroxy carboxylic acid in the present preparation is protonated. In case the acid contains more than one carboxylic group these percentages are to be applied to the total number of carboxylic groups. In order to ensure that the carboxylic acids remain protonated even when entering the mildly acidic intestinal tract, it may be advantageous to include an acidic buffer with a buffer pH at which the acid is largely protonated.

Best results are obtained with the present method if the preparation is administered no more than 60, preferably no more than 30 minutes before or after consumption of a foodstuff containing a significant amount, e.g. at least 20 g, of digestible carbohydrates. Thus the α-hydroxy carboxylic acid is allowed to move through the intestine together with the foodstuff, thereby effectively preventing enzymatic digestion of the saccharides contained therein, and simultaneously preventing dehydration.

The term digestible carbohydrates as used herein includes carbohydrates which can be absorbed directly by the intestine of the mammal as well as carbohydrates which are readily degraded within the intestine to such directly absorbable carbohydrates. Carbohydrates that are readily degraded within the intestine are those carbohydrates that can be digested by one or more of the salivatory, pancreatic or brush border enzymes of a given mammal. In case of humans these enzymes include glucoamylase (glucosidase), isomaltase, α-limit dextrinase, sucrase, lactase, pancreatic amylase and/or salivatory amylase.

The present method aims to inhibit intestinal carbohydrate absorption. Inhibition of intestinal carbohydrate absorption within the context of this invention refers specifically to a decrease of the intestinal enzyme activity that is associated with the hydrolysis of di-, tri-, oligo- and polysaccharides. Thus the present method leads to a decreased net absorption of monosaccharides from dietary digestible carbohydrates or to an absorption of monosaccharides over an increased surface area of the small intestine (i.e. absorption spread out over a longer period of time).

The present method is particularly suitable for (prophylactically) treating obesity as the reduction in carbohydrate absorption will usually also lead to a reduction in production of body fat. Another advantageous application of the method is its use for suppressing fluctuations in blood glucose levels, which is particularly beneficial for diabetics. Suppression of blood glucose fluctuations, and particularly the blood glucose ‘peaks’, is also of benefit for obese people as the resulting gradual absorption of carbohydrates usually leads to less body fat formation than is observed for rapid absorption of the same amount of carbohydrates.

The dietetic preparation according to the invention preferably contains α-hydroxy carboxylic acid component in an amount equivalent to at least 25 mg, more preferably equivalent to between 50 and 3000 mg, and most preferably equivalent to between 200 and 2000 mg citric acid.

The combination of α-amylase inhibitors and α-glucosidase inhibitors provide a potent blend of carbohydrase inhibitor. Such combinations are known in the art, however these combinations exhibit pronounced side effects, particularly when compared to a single carbohydrase inhibitors. These side effects include severe diarrhea, dehydration, flatulence and loss of intestinal fluids (see above). Such adverse side effects are observed to a much lower degree when the present method is employed, i.e. using a preparation containing an effective amount of α-hydroxy carboxylic acid component.

The supplementary intestinal carbohydrase inhibitor, preferably α-glucosidase inhibitor, to be used in combination with citric acid is preferably derived from plant material, preferably herbal plant material. The plant derived material used preferably comprises polyphenols. More preferably the plant derived material is an extract of a plant material in which the content of polyphenols is increased compared to the content of polyphenols naturally occurring in stems, leafs, roots and/or seeds of the same plant material.

Epimedium

According to a preferred embodiment of the present invention, the carbohydrase inhibitor co-administered with the α-hydroxy carboxylic acid is Epimedium plant material, preferably Epimedium brevicorum plant material. It was surprisingly found by the present inventors that Epimedium plant material inhibits intestinal carbohydrase. Hence, in a particularly preferred embodiment, the present invention provides a dietetic preparation comprising a combination of the α-hydroxy carboxylic acid and Epimedium plant material. This dietetic preparation is particularly effective in a method for the reduction of intestinal carbohydrate absorption, with the additional benefit of producing significantly less side effects, such as flatulence and diarrhea, compared to the use of α-hydroxy carboxylic acid alone. In a further preferred embodiment, a solvent extract of Epimedium brevicorum is used in the present method.

The Epimedium plant material is preferably administered in a daily amount of 10 mg to 5 g, preferably in a daily amount of 50 mg to 1000 mg.

preparation contains between 10 and 50 wt. % of the carbohydrate absorption inhibitor. More preferably the present preparation contains between 10 and 80 wt. % of plant polyphenols.

Tablets and equivalent solid and semi-solid oral dosage forms can suitably contain excipients such as hydroxypropylmethyl cellulose, other cellulosic materials, starch, polyvinyl-pyrrolidine, lactose and other sugars, starch, dicalcium phosphate, starch polymers, stearates, talc etc.

In yet another embodiment the present invention relates to a kit containing at least 10 dosage units comprising a dietetic preparation according to the invention, wherein the weight of the individual dosage units is between 0.3 and 10 g and said dosage units contain the α-hydroxy carboxylic acid component in an amount equivalent to between 100 and 2500 mg citric acid.

Carbohydrase Inhibitors

The dietetic preparation used in the present method may advantageously comprise one or more known carbohydrase enzyme inhibitors, since these inhibitors may complement the desirable effect of the α-hydroxy carboxylic acid component. In a preferred embodiment of this invention the dietetic preparation for inhibition of intestinal carbohydrate absorption further comprises a second carbohydrase inhibitor, preferably an intestinal α-glucosidase inhibitor, in an amount effective to provide synergistic action besides the α-amylase inhibition by the α-hydroxy carboxylic acid. Co-administration of the α-hydroxy carboxylic acid component and a second carbohydrase inhibitor (other than the α-hydroxy carboxylic acid component) offers the benefit of less side effects, such as flatulence and diarrhea, compared to the use of α-hydroxy carboxylic acid alone. Exemplary and preferred carbohydrase inhibitors used in accordance with the present invention include Phaseolus vulgaris (phaseolamin), roselle tea, lotus, arabinose, inosine, adenosine, evening primrose extract, banaba extract, Epimedium extract, indigestible dextrin and polyphenols.

The combination of α-amylase inhibitors and α-glucosidase inhibitors provide a potent blend of carbohydrase inhibitor. Such combinations are known in the art, however these combinations exhibit pronounced side effects, particularly when compared to a single carbohydrase inhibitors. These side effects include severe diarrhea, dehydration, flatulence and loss of intestinal fluids (see above). Such adverse side effects are observed to a much lower degree when the present method is employed, i.e. using a preparation containing an effective amount of α-hydroxy carboxylic acid component.

The supplementary intestinal carbohydrase inhibitor, preferably α-glucosidase inhibitor, to be used in combination with citric acid is preferably derived from plant material, preferably herbal plant material. The plant derived material used preferably comprises polyphenols. More preferably the plant derived material is an extract of a plant material in which the content of polyphenols is increased compared to the content of polyphenols naturally occurring in stems, leafs, roots and/or seeds of the same plant material.

Epimedium

According to a preferred embodiment of the present invention, the carbohydrase inhibitor co-administered with the c-hydroxy carboxylic acid is Epimedium plant material, preferably Epimedium brevicorum plant material. It was surprisingly found by the present inventors that Epimedium plant material inhibits intestinal carbohydrase. Hence, in a particularly preferred embodiment, the present invention provides a dietetic preparation comprising a combination of the α-hydroxy carboxylic acid and Epimedium plant material. This dietetic preparation is particularly effective in a method for the reduction of intestinal carbohydrate absorption, with the additional benefit of producing significantly less side effects, such as flatulence and diarrhea, compared to the use of α-hydroxy carboxylic acid alone. In a further preferred embodiment, a solvent extract of Epimedium brevicorum is used in the present method.

The Epimedium plant material is preferably administered in a daily amount of 10 mg to 5 g, preferably in a daily amount of 50 mg to 1000 mg.

Polyphenols

According to a preferred embodiment of the current invention, the carbohydrase inhibitor, preferably α-glucosidase inhibitor, are plant derived polyphenols, selected form the group consisting of catechins or derivatives thereof, anthocyanidins, proanthocyanidins, procyanidin and cyanidin, which are exemplary and preferably obtained from green tea (Camellia sinensis) or grape (Vitis vinifera). Preferably such plant extracts have a significant content of polyphenols, increasing the effectiveness as an intestinal carbohydrase inhibitor. However, oral intake of polyphenols, especially extracts, will result in a decreased absorption of water in the intestine, resulting in many cases in diarrhea and loss of intestinal fluid, potentially followed by proliferation and growth of undesirable intestinal bacteria and damage to the intestinal cells. Such adverse side effects of polyphenol ingestion, especially compositions having high polyphenol content, will be prevented by the co-administration of α-hydroxy carboxylic acid component.

Herbal extracts comprising polyphenols are known in the art. Most suitable for use in the method and preparation according to the present invention, are extracts comprising more than about 10 wt. % polyphenols based on the dry weight of the plant extract, preferably above about 25 wt. % polyphenols even more preferably above about 50 wt % polyphenols and most preferably above about 75 wt % polyphenols.

Green Tea Extract

The dietetic preparation of the present invention may advantageously contain green tea extract as a source of polyphenols. Green tea catechins or derivatives thereof (including monomers, polymers or gallated monomers or polymers of catechin) have been described to inhibit the intestinal α-glucosidase enzyme (Matsui et al, Biosci Biotechnol Biochem 1996 December; 60(12):2019-22). Additionally green tea has been ingested for centuries by human beings and can therefore be regarded as very safe.

Preferably green tea extracts used in the preparation according to the invention comprise more than 20 wt. %, more preferably more than 40 wt. % catechins expressed as epigallocatechin gallate based on the total dry weight of the green tea extract, so as to provide sufficient carbohydrase inhibitory action. Preferably the green tea extract is administered in a daily amount of between 10 mg and 5 g, more preferably in a daily amount of between 50 mg and 2.5 g.

Grapeseed Extract

To further stimulate the action of α-hydroxy carboxylic acid or the combination of such acid and polyphenols (e.g. green tea polyphenols), grape polyphenols can be added to the formulation. Grape polyphenols are preferably obtained from the seeds. Suitable for use in the composition according to the invention is grape seed powder, however, according to a preferred embodiment grape seed (powder) extract is used.

Grape seed powder or extract preferably comprises an effective amount of grape polyphenols, preferably one or more selected from anthocyanidins, proanthocyanidins, procyanidin and cyanidin. The grapeseed powder or extract preferably comprises more than about 10 wt. % grape polyphenols based on the dry weight of the grape seed powder or extract, preferably more than about 25 wt. % polyphenols, even more preferably more than about 50 wt. % polyphenols, most preferred above about 75 wt. % polyphenols. Preferably the grape seed powder or extract is administered in a daily amount of between 10 mg and 5 g, more preferably in a daily amount of between 50 mg and 2.5 g.

Monosaccharide Absorption Inhibitor

Advantageously, the preparation according to the present invention comprises a component capable of inhibiting monosaccharide uptake in the intestine. Such a component, when used alone, can also produce the adverse side effects mentioned above, i.e. diarrhea, flatulence etc. When used in combination with α-hydroxy carboxylic acid component such undesirable effects will be reduced or prevented.

The action of the monosaccharide uptake inhibitor will further enhance the effects of inhibition of the carbohydrate absorption and/or increase the intestine surface area across which the carbohydrate is absorbed. Thus the co-administration of a monosaccharide uptake inhibitor will increase the performance of the present preparation. The inhibition of uptake of monosaccharides by the monosaccharide uptake inhibitor, increases the monosaccharide/digestible carbohydrate ratio, thereby decreasing the conversion rate of digestible carbohydrates to monosaccharides and thus providing the α-hydroxy carboxylic acid and other carbohydrase inhibitors the opportunity to further inhibit the carbohydrase activity.

The substances capable of inhibiting monosaccharide uptake used in a preferred embodiment according to this invention are capable of decreasing transport of monosaccharide over the intestinal wall without the necessity for a decrease in intestinal glucose concentration. However, excess content of monosaccharide uptake inhibitor in the dietetic preparation according to the invention might interfere with the rehydration action of the α-hydroxy carboxylic acid component. Monosaccharide uptake inhibitors which may advantageously be employed in the present method include fibrous and non-fibrous monosaccharide uptake inhibitors.

In case non-fibrous monosaccharide uptake inhibitors are employed, the weight ratio monosaccharide inhibitor to α-hydroxy carboxylic acid component is between about 10:1 and 1:250, more preferably between 1:1 and 1:100, and most preferably between 1:5 and 1:50. Preferably the non-fibrous monosaccharide uptake inhibitor is of plant origin Preferably such a substance is of a plant origin, of which the safety has been well established. Exemplary non-fibrous monosaccharide uptake inhibitors are peppermint (oil), procyanidin, galloyl residues or can be obtained from Gymnema species, Azadirachta indica, Eugenia uniflore, Ginseng radix, soy. An especially preferred compound for such action to be used in the preparation according to the invention is gymnemic acid. This substance can, for example, be found in plants of the species Gymnema, e.g. Gymnema sylvestre. According to a further preferred embodiment the composition comprises at least 5 wt. %, more preferably at least 10 wt. % and most preferably at least 20 wt. % gymnemic acid calculated on dry weight of the monosaccharide uptake inhibitor.

According to a further preferred embodiment of the invention the dietetic preparation comprises a fibrous monosaccharide uptake inhibitor, such as indigestible plant carbohydrates, particularly plant fibres. Preferably the fibrous monosaccharide uptake inhibitor comprises an effective amount of soluble fibre selected from the group consisting of pectin, guar gum, Konjak mannan, locust bean gum, oat fibre, inulin and mixtures thereof.

Use

The preparation according to the invention can be used advantageously by subjects having the desire or need to reduce carbohydrate absorption, or desiring to decrease fluctuations in blood glucose level. The preparation may be used as such in weight managament programs or can be included in compositions designed for weight management, for athletes having the desire to decrease carbohydrate absorption and for preventing the adverse side effects of craving, etc.

Compositions that aim to meet the above objectives are known in the art and are often referred to as “carbohydrate cutters”, “carb-cutters”, “carbohydrate blockers”, “carb blockers”, compositions providing balanced and/or healthy blood sugar levels, (high) protein bars and the like.

### EXAMPLES

### Example 1

A oral nutritional supplement in the form of a capsule comprising 500 mg citric acid, to be administrated before, during or shortly after carbohydrate containing meal or snack.

### Example 2

A oral nutritional supplement in the form of a capsule comprising

200 mg green tea extract (75 wt. % polyphenols based on the weight of the green tea extract)

500 mg citric acid

to be administrated before, during or shortly after carbohydrate containing meal or snack.

### Example 3

A oral nutritional supplement in the form of a capsule comprising

250 mg Gymnema sylvestre extract (25 wt. % gymnemic acid based on the weight of the gymnema sylvestre extract)

500 mg citric acid

to be administrated before, during or shortly after carbohydrate containing meal or snack.

### Example 4

A oral nutritional supplement in the form of a capsule comprising

250 mg Gymnema sylvestre extract (25 wt. % gymnemic acid based on the weight of the gymnema sylvestre extract)

100 mg green tea extract (75 wt. % polyphenols based on the weight of the green tea extract)

50 mg Grapeseed extract (90 wt. % polyphenols based on the weight of the grapeseed extract)

300 mg citric acid

to be administrated before, during or shortly after carbohydrate containing meal or snack

### Example 5

A dietetic food preparation in the form of a coated tablet, to be ingested within 45 minutes prior to the consumption of a foodstuff containing a significant amount of digestible carbohydrates, said tablet comprising:

300 mg citric acid

1000 mg Konjak mannam

### Example 6

A placebo controlled, double-blind, randomized, parallel study was conducted to evaluate the tolerance of a composition containing citric acid, grape seed extract, green tea extract and Gymnema Sylvestre leave extract.

Study Population

Volunteers were recruited in Wageningen (the Netherlands) and surroundings. Posters at several locations at the university and student flats and advertisements in local newspapers were used. Inclusion criteria for study participation were: Body Mass Index (BMI) between 20 and 24.9 kg/M², age between 18 and 45 years. Exclusion criteria were: diabetes mellitus, chronical intestinal diseases or related symptoms (present and history), acute diarrhea during the previous month, constipation, use of medication affecting the gastrointestinal tract (e.g. antibiotics, laxatives), unusual dietary habits (e.g. specific diets, vegans), pregnancy or intention to get pregnant.

During the screening visit body weight and body height were determined. Body weight was measured to the nearest 0.1 kg using a precision scale without shoes with subjects dressed in light clothing. Height was determined to the nearest cm without shoes. BMI was calculated from weight and height: weight(kg)/(height(m))². Depending on this result it was decided whether the subject could participate in the study.

Seventeen healthy subjects (6 males, 11 females) in the age of 27±5 years (mean±SD) and BMI. 22.2±1.8 kg/M² (mean±SD) were recruited. The study was explained by the investigator. All subjects signed informed consent forms prior to their entry into the study.

Study Design

Participants were randomized over 2 groups. Each group received Carbcutter or Placebo (ingredients of a single dose of Carbcutter and Placebo are provided in Table 1 below). Tolerance of the product was determined for two weeks.

On the first study day, after a 12 h overnight fast, a blood sample was obtained to measure safety parameters. To test the tolerance of the products, the subjects were asked to consume one of the products (Carbcutter or Placebo) for two weeks. A single dose of the products was ingested with each of the two main meals; i.e. two times a day. At the end of each day, the subjects were asked to fill in the provided questionnaire about gastrointestinal complaints, stool frequency and stool consistency. At the end of the two weeks of tolerance, after a 12 h overnight fast, body weight was measured and a blood sample was taken to measure safety parameters. As safety endpoints, changes in liver and kidney function before and after the tolerance period were determined.

Test Products

The product ingredients are specified in Table 1

TABLE 1

| | Supplement | Placebo | | |
|---|---|---|---|---|
| Ingredient | (g) | (g) | Characteristics | Supplier |
| Citric acid | 300 | 0 | | Citric acid anhydrous |
| | | | | Citrique Belge N.V. |
| Green tea leave | 100 | 0 | Polyphenols 95.7% | P. L. Thomas & Co., |
| extract | | | Epigallo catechin gallate | Inc. |
| | | | 35% | |
| Grape seed extract | 50 | 0 | Phenolics (gallic acid | Polyphenolics |
| | | | equivalents) 98.7% | |
| | | | (min 90 g GAE/100 g) | |
| Gymnema Sylvestre | 200 | 0 | Gymnemic acid 28.24% (25-30%) | Sabinsa corporation |
| leave extract | | | | |
| Calcium Carbonate | 0 | 650 | | |
| Total | 650 | 650 | | |

Serving Size

1 capsule per meal. Capsules had to be taken with the two main meals, i.e. 2 capsules a day.

Questionnaire

Gastrointestinal complaints concerning flatulence, bloating, abdominal pains or cramps, eructation, nausea, vomiting and stomach pains or cramps were rated on a 5-point scale. Stool consistency was rated on a 5-point scale based on the scale by Heaton et al. (Gut 1992;33(6):818-24): watery-soft, pudding like-soft, snake like-dry, cylindric-dry, hard pellets. Stool frequency was also recorded. Other adverse effects could be recorded in the questionnaires.

Biochemical Measurements

As safety parameters the following blood parameters were measured at the beginning and at the end of the study: aspartate aminotransferase (AST), alanine aminotransferase (ALT), lactic acid dehydrogenase (LDH), creatinine, gamma-glutamyl transferase (GGT), alkaline phosphatase, and urea nitrogen (BUN). Blood samples were collected in clotting tubes and centrifuged after clotting. Plasma samples were analyzed according to standard laboratory methods.

Statistical Analysis

Differences between groups for the questionnaire were analyzed using the non-parametric Mann-Whitney U test for unpaired observations. For the categorical data with two categories, a comparison between the two treatment groups was performed using the Fishers' exact test. The safety laboratory values were statistically tested using the two-sample Mann-Whitney U test for unpaired samples. Statistical differences were assumed when P<0.05.

Results

Questionnaire

Following administration of the Carbcutter, no significant effects on bloating, abdominal pain, stomach ache, eructations, nausea, frequency of stools/day, consistency of all the stools, general physical well being and vomiting were observed compared to placebo.

Also no other adverse events were reported. For Results of the questionnaire, the mean scores over 14 days for the two groups results, see Table 2.

TABLE 2

| | Carbcutter^{a} | Placebo^{a} | P-value |
|---|---|---|---|
| Flatulence^{b} | 1.7 | 1.7 | 0.650^{f} |
| Abdominal pain^{b} | 1.2 | 1.3 | 0.395^{f} |
| Bloating^{b} | 1.2 | 1.6 | 0.076^{f} |
| Stomach ache^{b} | 1.1 | 1.2 | 0.892^{f} |
| Eructations^{b} | 1.1 | 1.0 | 0.239^{f} |
| Nausea^{b} | 1.1 | 1.2 | 0.445^{f} |
| Frequency of stools/day | 1.4 | 1.5 | 0.941^{f} |
| Consistency of all the stools^{c} | 3.2 | 3.3 | 0.459^{f} |
| General physical well being^{d} | 7.9 | 7.3 | 0.230^{f} |
| Vomiting^{e} | 2.0 | 2.0 | 0.452^{g} |
| Other adverse events^{e} | 1.8 | 1.7 | 0.427^{g} |
| Use medication^{e} | 2.0 | 1.9 | 0.292^{g} |

^{a}Means of all variables were given for the tolerance period of 14 days.
^{b}range 1 to 5:1 = not at all, 5 = continuous
^{c}1 = watery, 2 = pudding like-soft, 3 = snake like-soft, 4 = cylindric-dry, 5 = hard pellets.
^{d}range 1 to 10:1 = very bad, 10 = very good
^{e}1 = yes, 2 = no
^{f}Differences between groups were analyzed using the non-parametric Mann-Whitney U test for unpaired observations (with corrections for ties if applicable). P < 0.05 was considered to be significant.
^{g}Differences between groups were analyzed using the Fisher's exact test. P < 0.05 was considered to be significant.

Safety Parameters

As safety endpoints, changes in liver function and kidney function before and after the tolerance period were determined. In Table 3 mean changes in kidney and liver enzyme levels in the two groups±SEM are shown. Differences between the two groups were not significantly different except for Urea nitrogen. Probably this was due to the significant difference already found at baseline characteristics for Urea nitrogen.

TABLE 3

| | Carbcutter | Placebo | P-value |
|---|---|---|---|
| ASAT (U/L) | 1.3 ± 2.3 | −1.7 ± 1.2 | 0.300 |
| ALAT (U/L) | 5.4 ± 1.9 | 5.7 ± 0.9 | 0.905 |
| LD (U/L) | 41.7 ± 53 | −36.4 ± 8.7 | 0.133 |
| Creatinine (μmol/L) | −6.6 ± 1.7 | −5.7 ± 3.0 | 0.813 |
| GGT (U/L) | 1.5 ± 0.6 | 0.3 ± 0.3 | 0.206 |
| Alkaline phosphatase (U/L) | 0.1 ± 3.5 | 2.7 ± 1.6 | 0.651 |
| Urea nitrogen (mmol/L) | 0.16 ± 0.2 | −0.7 ± 0.3 | 0.040 |

### CONCLUSION

Following administration of the supplement, no significant effects on flatulence, bloating, abdominal pain stomach ache, eructations, nausea, frequency of stools/day, stool consistency, general physical well being and vomiting were observed compared to placebo. So administration of the product for 14 days was well-tolerated.

1. Use of citric acid component in the manufacture of a dietetic preparation for use in a method for inhibiting intestinal absorption of carbohydrates in a mammal, which method comprises orally administering the dietetic preparation to such mammal, said preparation containing citric acid component selected from the group consisting of citric acid, citric acid salts, citric acid estes and mixtures thereof in an amount equivalent to at least 1 wt. % citric acid, so as to provide citric acid component in an amount which is therapeutically effective to achieve inhibition of intestinal absorption of carbohydrate.

2. Use according to claims 1, wherein the citric acid component is citric acid.

3. Use according to claims 1 or 2, wherein the method comprises providing the citric acid component in a daily amount equivalent to at least 0.5 mg citric acid per kg of bodyweight.

4. Use according to any one of the preceding claims, wherein the preparation is administered no more than 60 minutes before or after consumption of a foodstuff containing at least 20 g of digestible carbohydrates.

5. Use according to any one of the preceding claims, wherein the preparation comprises less than 60 wt. % digestible carbohydrates based on dry weight of the preparation.

6. Use according to any one of the preceding claims, wherein the method comprises administration of the preparation in dosage units of between 0.1 and 100 g.

7. Use according to any one of the preceding claims, wherein the preparation contains citric acid component in an amount equivalent to between 50 and 3000 mg citric acid.

8. Use according to any one of the preceding claims, wherein the preparation additionally contains an intestinal carbohydrase inhibitor selected from the group consisting of Phaseolus vulgaris (phaseolamin), roselle tea, lotus, arabinose, inosine, adenosine, evening primrose extract, banaba extract, Epimedium extract, indigestible dextrin, polyphenols and mixtures thereof.

9. Use according to claim 8, wherein the preparation additionally contains Epimedium extract.

10. Use according to any one of the preceding claims, wherein the preparation additionally contains a monosaccharide uptake inhibitor selected from the group consisting of pectin, guar gum, Konjak mannan, locust bean gum, oat fibre, inulin and mixtures thereof.

11. Use according to claim 10, wherein the preparation contains inulin.

12. Dietetic preparation in the form of an oral dosage unit of between 0.1 and 100 grams, said preparation containing between 2 and 90 wt. % citric acid component between 1 and 80 wt. % of a carbohydrate absorption inhibitor selected from the group consisting of polyphenols, gymnemic acid, Epimedium plant material and mixtures thereof and between 97 and 9 wt. % of pharmaceutically acceptable excipient.

13. Dietetic preparation according to claim 12, comprising Epimedium plant material.

14. Dietetic preparation according to claims 12 or 13, wherein the oral dosage unit is a tablet or capsule of between 0.3 and 10 grams.

15. Dietetic preparation according to any one of the claims 12-14, wherein the preparation contains citric acid component in an amount equivalent to at least 25 mg, preferably equivalent to between 50 and 3000 mg citric acid.

16. Dietetic preparation according to any one of the claims 12-15, wherein the preparation comprises less than 60 wt. % digestible carbohydrates calculated on dry weight of the preparation.

17. Dietetic preparation according to any one of the claims 12-16, wherein the preparation contains between 10 and 80 wt. % plant polyphenols.

18. Dietetic preparation according to any one of the claims 12-17, wherein the preparation additionally contains a monosaccharide uptake inhibitor, said absorption reducing component being selected from the group consisting of pectin, guar gum, Konjak mannan, locust bean gum, oat fibre, inulin, indigestible dextin and mixtures thereof.

19. Dietetic preparation according to any one of the claims 12-18, wherein the preparation contains at least 10 wt. % α-hydroxy carboxylic acid component, less than 50 wt. % water and less than 10 wt. % digestible carbohydrates.

20. Kit containing at least 10 oral dosage units comprising the dietetic preparation according to claim 12, wherein the weight of the individual dosage units is between 0.3 and 10 g and said dosage units contain the citric acid component in an amount equivalent to between 100 and 2500 mg citric acid.
